(19) United States
(12) Patent Application Publication
(10) Pub. No.: US 2016/0045407 A1
(43) Pub. Date: Feb. 18, 2016

(54) DENTRIFICE WITH INCREMENTAL CHEMISTRIES

(71) Applicant: The Procter & Gamble Company, Cincinnati, OH (US)

(72) Inventor: Paul Albert Sagel, Mason, OH (US)

(21) Appl. No.: 14/826,653

(22) Filed: Aug. 14, 2015

Related U.S. Application Data
(60) Provisional application No. 62/038,185, filed on Aug. 15, 2014.
(60) Provisional application No. 62/038,335, filed on Aug. 17, 2014.

(51) Int. Cl. A61K 8/21; A61K 8/22; A61Q 11/00

(52) U.S. Cl. CPC A61K 8/21; A61Q 11/00; A61K 8/22; A61K 2800/87

(57) ABSTRACT

A dentifrice composition is provided. The dentifrice composition can include a multi-stage composition adapted to provide incremental chemistries during use.

### FIELD OF THE INVENTION

Dentifrice compositions are provided, such as, for example, a dentifrice composition adapted to provide incremental chemistries during use.

### BACKGROUND OF THE INVENTION

Many people in developed countries are aware of the need for preventative dental care, including twice-daily tooth brushing with fluoridated toothpaste, daily flossing, and bi-annual dental visits; however, compliance with such a regimen is often poor. Some users, for example, brush only once daily for less than the recommended two-minute time period and rarely floss. Over time, this allows plaque to build up on the user's teeth faster than the user brushes it away.

After a few days, the plaque turns into tartar, which must be removed by the dentist. As the tartar continues to build, the eventual dental visit to remove the tartar becomes even longer and more unpleasant for the user, which can reinforce the user's distaste for dental care in general. In addition, the user may begin to experience gum inflammation, bleeding, and pain, which can further reduce the user's desire to brush and floss frequently.

Even users that are generally compliant with twice-daily brushing may not brush for a full two minutes, as users are notoriously inaccurate at estimating the time they spent brushing. For example, users may believe they have brushed for a full two minutes when they actually have only spent 45-60 seconds brushing. Some users may use a timer to monitor their brushing habits, however, the two minutes can still feel like an eternity to the user as the clock counts down the seconds.

Because most users are using a toothpaste and toothbrush for at least some time each day, toothpaste and toothbrush design can have a significant impact on a user's oral health. Unfortunately, effective oral care agents, such as fluoride and peroxide, are often unstable and difficult to formulate into an effective toothpaste, particularly since many users do not brush long enough or with the proper technique necessary to realize the maximum benefits of the toothpaste during use. In addition, users are often unwilling to add additional steps or to modify their brushing technique to match a new toothpaste or other oral health care regimen.

As such, there remains a need for a dentifrice, such as a toothpaste, that is adapted to typical brushing habits. There also remains a need for a dentifrice that can effectively deliver one or more oral care agents, particularly during short brushing times or with imperfect brushing technique. There further remains a need for a dentifrice that continues to be effective after brushing.

### SUMMARY OF THE INVENTION

A dentifrice composition comprising a multi-stage composition adapted to provide incremental chemistries during use is provided. The multi-stage composition can have a first stage comprising a fluoride agent, a second stage comprising a residue of the fluoride agent obtained after application and expectoration of the first stage, and a third stage comprising a mixture of the residue of the fluoride agent and a bleaching agent, wherein the bleaching agent is added to the fluoride agent after the expectoration of the first stage.

A dentifrice composition comprising a multi-stage composition having incompatible first and second effective agents is also provided. The first effective agent can include a fluoride agent. The first effective agent can be contained in a first container and can be applied to a user's mouth and expectorated to leave a fluoride residue. The second effective agent can include a bleaching agent. The second effective agent can be contained in a second, separate container. In addition, the second effective agent can be applied to a user's mouth to mix with the fluoride residue; wherein the fluoride reside is detectable during application of the second effective agent.

Further provided is a dentifrice composition comprising a mixture of a residue of an unmitigated fluoride agent and a dose of a bleaching agent. The residue is obtained after a dose of the fluoride agent is applied to the teeth, brushed with a toothbrush, and expectorated without rinsing.

### BRIEF DESCRIPTION OF THE DRAWINGS

FIG. 1 illustrates an exemplary dentifrice with incremental chemistry in accordance with the present invention.

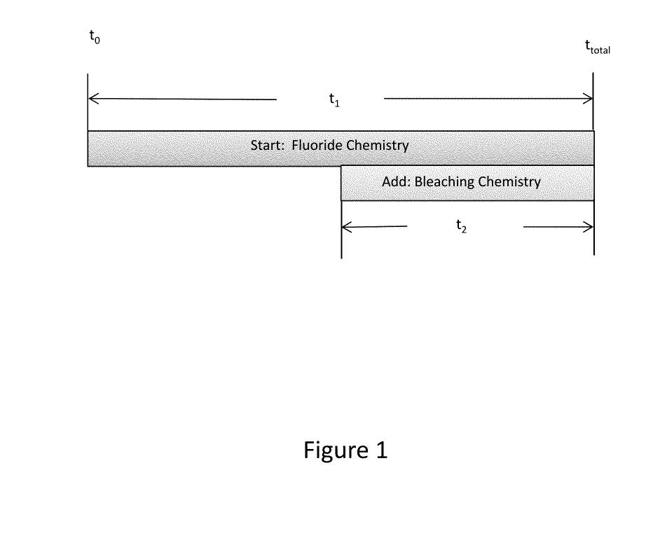

### DETAILED DESCRIPTION OF THE INVENTION

Dentifrices with incremental chemistries are provided. Surprisingly, such dentifrices are adapted to be effective with shortened or infrequent brushing practices. In addition, such dentifrices can provide extended effectiveness after use.

The dentifrice compositions are generally multi-stage compositions adapted to provide incremental chemistries during use. In one example, the multi-stage composition can have a first stage including a fluoride agent, a second stage including a residual amount of the fluoride agent obtained after application and expectoration of the first stage, and a third stage including a mixture of the residual fluoride agent and a bleaching agent. The bleaching agent is added after expectoration of the first stage. In addition, the user does not rinse after expectoration of the first stage such that the residue of the first stage is present in the user's mouth during application of the bleaching agent.

The incremental application of such chemistries such that residual fluoride can mix with the bleaching agent in the user's mouth, but not before, results in extended effectiveness, particularly overnight. In some examples, such incremental chemistries also can reduce or eliminate the negative aesthetic characteristics provided by the fluoride agent, such as, for example, staining or unpleasant taste. In some examples, the user can experience staining and/or unpleasant taste during the first stage and second stage; however, such negative aesthetic characteristics can be neutralized during the third stage.

The fluoride chemistry and bleaching chemistry each can be used for a shortened brushing time, such as, for example, from about 45 seconds to about 1 minute. Surprisingly, because the user is continually changing activities during the brushing period, such as, for example, brushing for a short time, expectorating, adding the bleaching agent after expectoration of the first stage, and then brushing for a second short time, the user can continue to use habitual quick brushing techniques and may be less likely to become bored or distracted during the brushing period.

In one example, the fluoride agent is stannous fluoride and the bleaching agent is hydrogen peroxide. In another example, the fluoride agent is sodium fluoride and the bleaching agent is hydrogen peroxide. The fluoride agent does not contain the bleaching agent

The fluoride agent and the bleaching agent can be contained in separate containers. In one example, the fluoride agent is contained in a first container with a first shape or orientation and the bleaching agent is contained in a second container with a second shape or orientation that is different from the first shape or orientation. This allows the user to quickly identify and grasp the bleaching agent after expectoration of the fluoride agent. In addition, this facilitates the user applying the agents in the correct order, instead of mistakenly applying the bleaching agent during the first stage and the fluoride agent during the third stage, which will not provide the user with the intended experience.

In one example, the fluoride chemistry can provide health benefits including antibacterial benefits and can contain an abrasive, such as silica, and a fluoride salt, such as stannous fluoride. Residue of the fluoride salt can remain in the user's mouth after brushing with the fluoride chemistry and expectorating, but not rinsing. The residual fluoride is then available to mix with the bleaching chemistry once the bleaching chemistry is applied to the user's mouth. In one example, the fluoride chemistry does not contain a bleaching agent. In another example, the fluoride chemistry does not contain a stain mitigation agent. The bleaching chemistry can provide cosmetic benefits including stain control, whitening and/or breath freshening benefits and can contain peroxide, such as hydrogen peroxide. In one example, the bleaching composition can be an oral care gel and does not contain a fluoride salt or an abrasive.

In one example, a user can dispense the fluoride chemistry onto a toothbrush and can proceed to apply the fluoride chemistry to the oral cavity as part of a brushing regimen for a predetermined period of time. In one example, the fluoride chemistry can be used for about one minute. In one example, the fluoride chemistry can foam during use. After use of the fluoride chemistry, the user can expectorate the fluoride chemistry. However, the user should not rinse. Then, the user dispenses the bleaching chemistry onto the toothbrush and applies the bleaching chemistry to the oral cavity as part of the brushing regimen for a predetermined amount of time. In one example, the fluoride chemistry can be used for about one minute. Then, the user can expectorate and can rinse her mouth with water. In one example, the bleaching chemistry can be applied to a toothbrush or the oral cavity within about 5, 10, 15, 30, 45, 60, 120, 180, 240, 300, 360, 420, seconds or 10, 15, or 20 minutes of the fluoride chemistry being applied to a toothbrush or the oral cavity or expectorated from the oral cavity, provided that sufficient fluoride residue remains in the oral cavity during application of the bleaching chemistry to provide the incremental benefits of the multi-stage composition.

Such dentifrices with incremental chemistries can also provide a unique sensory experience for the user during the brushing process. This unique sensory experience can be enhanced when the products are applied in a multi-stage process, where a composition containing a fluoride salt, such as stannous fluoride, is applied first, expectorated without rinsing, and then a composition containing a hydrogen peroxide is applied while sufficient residue from the fluoride salt remains in the user's mouth. In particular, while using the fluoride composition, many users describe the fluoride composition as warm and clean feeling in the mouth, however, consumers can also find the fluoride composition astringent and dry. Consumers commented that when just the fluoride oral care composition is used, the regimen can feel unfinished. However, the bleaching composition, which contains peroxide, can provide a cool, smooth, lasting taste, that can be enjoyable to the user. Consumers can find that their mouth feels fresh and clean all day and many consumers believe that this feeling persists even after they eat and drink. After brushing, some consumers described that their teeth and mouth felt glossy, shiny, vibrant, radiant, and smooth.

However, if a user rinses her mouth between application of the fluoride chemistry and the bleaching chemistry, or otherwise alters the regimen, the same unique experience may not be provided. For instance, the bleaching chemistry may feel harsh due to the peroxide and the fluoride composition may taste overly astringent and the flavor of food and beverages may be negatively impacted. Also, if the incremental chemistry is modified or rearranged, the ratio of fluoride chemistry to bleaching chemistry upon administration of the bleaching chemistry will be disrupted. By “oral care composition”, as used herein, is meant a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, mouth rinse, mousse, foam, mouth spray, lozenge, chewable tablet, chewing gum, tooth whitening strips, floss and floss coatings, breath freshening dissolvable strips, or denture care or adhesive product. The oral care composition may also be incorporated onto strips or films for direct application or attachment to oral surfaces.

The term “dentifrice”, as used herein, includes tooth or subgingival -paste, gel, or liquid formulations unless otherwise specified. The dentifrice composition may be a single phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side. The term “dispenser”, as used herein, means any pump, tube, or container suitable for dispensing compositions such as dentifrices.

The term “teeth”, as used herein, refers to natural teeth as well as artificial teeth or dental prosthesis.

As used herein, the word “include,” and its variants, are intended to be non-limiting, such that recitation of items in a list is not to the exclusion of other like items that may also be useful in the materials, compositions, devices, and methods of this invention.

As used herein, the word “or” when used as a connector of two or more elements is meant to include the elements individually and in combination; for example X or Y, means X or Y or both.

All percentages and ratios used hereinafter are by weight of total composition, unless otherwise indicated. All percentages, ratios, and levels of ingredients referred to herein are based on the actual amount of the ingredient, and do not include solvents, fillers, or other materials with which the ingredient may be combined as a commercially available product, unless otherwise indicated.

All measurements referred to herein are made at 25° C. (i.e. room temperature) unless otherwise specified.

The composition can contain, consist of, or consist essentially of, the essential elements and limitations of the invention described herein, as well as any additional or optional ingredients, components, or limitations described herein or otherwise useful in oral care compositions. Actives and other ingredients may be categorized or described herein by their cosmetic benefit, therapeutic benefit, or their postulated mode of action or function. However, it is to be understood that the active and other ingredients useful herein can, in some instances, provide more than one cosmetic benefit, therapeutic benefit, function, or can operate via more than one mode of action. Therefore, classifications herein are made for the sake of convenience and are not intended to limit an ingredient to the particularly stated function(s) or activities listed.

The fluoride composition may comprise a metal salt. Suitable metal salts include salts of copper (Cu), zinc (Zn), silver (Ag), tin (Sn), magnesium (Mg), iron (Fe), sodium (Na), and manganese (Mn) salts, or combinations thereof. Preferred salts include, without limitation, gluconates, chlorates, citrates, chlorides, fluorides, and nitrates, or combinations thereof. In some embodiments, the metal salt is sodium fluoride, sodium monofluorophosphate, stannous fluoride, or combinations thereof. In some embodiments, the metal salt is stannous fluoride. Sodium fluoride, sodium monofluorophosphate, and/or stannous fluoride, if used, may be included in the fluoride composition at 850 to 1,150 ppm theoretical total fluorine. Some metal salts which may be used in the present invention, such as zinc chloride, zinc citrate, copper gluconate, and zinc gluconate, are also associated with an off taste described as dirty, dry, earthy, metallic, sour, bitter, and astringent. See, for example, an article by Hu, Hongzhen, et al in Nature Chemical Biology (2009), 5 (3), Pages 183-190, entitled: Zinc Activates Damage-Sensing TRPA1 Ion Channels. In some embodiments, a metal salt associated with an off taste, such as zinc chloride, zinc citrate, copper gluconate, zinc gluconate, or combinations thereof, is used with a metal salt with recognized anti-caries activity, such as sodium fluoride, sodium monofluorophosphate, stannous fluoride, or combinations thereof.

The metal salt, if present, may provide anti-caries, reduced tooth sensitivity, stain reduction, combinations of these benefits, and/or other benefits. The fluoride composition may further comprise an abrasive for cleaning purposes. Abrasives are solid materials added to dentifrices to facilitate mechanical removal of dental plaque, debris, and/or stain from tooth surfaces. In some embodiments, the fluoride composition is a dentifrice, i.e., an abrasive-containing dosage form for delivering an anticaries substance to the teeth. In some embodiments, the fluoride composition is not a dentifrice.

The fluoride composition may comprise flavorants. The flavorants in conventional oral care compositions are generally selected and dosed to overcome any unpleasant taste or mouthfeel from the active ingredients (for oral health) and/or carrier ingredients (for suspending, homogenizing, and/or stabilizing the active ingredients in desired concentrations, which may vary by dosage form). In a preferred embodiment of the present invention, the fluoride composition contains only sufficient flavorant to counteract any distinctly distasteful experience that might discourage use of the fluoride composition entirely. In some embodiments, the fluoride composition comprises flavorants in an amount greater than 0% and less than about 2.00%, or less than about 1.60%, by weight of the composition. The flavorants may include sweeteners, such as saccharin, or natural flavors, such as extracts of mint or spearmint, or artificial flavors, or sensates that create a sensation of coolness or warmth in the mouth, or combinations thereof.

The fluoride composition may be delivered during the first stage. The first stage may have sub-stages. The first stage may comprise applying the composition to a dental hygiene device. The first stage may comprise using the dental hygiene device to apply the fluoride composition to the teeth and/or gums. The first stage may comprise expectorating. In some embodiments, the first stage does not include rinsing the mouth, as with water or mouthwash. In some embodiments, there is no rinsing, as with water or mouthwash, after the first stage, or no rinsing from the start of the brushing period until completion of the delivery of the chemistries (e.g., rinsing may be the final step in the entire delivery).

In some embodiments, for example, where the fluoride composition comprises a metal salt and the bleaching composition comprises an oxidizing agent, it may be less efficient to introduce the bleaching composition into the mouth without first expectorating. As a specific, non-limiting example, the fluoride composition may comprise stannous fluoride and the bleaching composition may comprise hydrogen peroxide. Stannous fluoride and hydrogen peroxide react readily, in a matter of seconds, so even if the first and bleaching compositions are introduced separately, their interaction in the mouth may promptly inactivate much of the stannous fluoride and hydrogen peroxide. However, it may be desirable to leave some amount of stannous fluoride in the mouth, e.g., by not rinsing after using the fluoride composition, so that the anti-caries, pro-gum health, and breath freshening effects of the stannous fluoride persist during the bleaching stage, even if at a lesser degree than during the fluoride stage, when no peroxide was present. Expectorating may reduce the amount of hydrogen peroxide precipitated by the stannous fluoride, while leaving some stannous fluoride on the teeth and/or gums for continued action. Similar benefits may be achieved with other combinations of actives in the first and bleaching compositions. Stannous fluoride and hydrogen peroxide are an important example because of the kinetics of the reaction between them. Further, by localizing the precipitation reaction to the surfaces of the teeth and gums, the precipitated salts may physically occupy dentinal tubules, thereby reducing the transmission of sensitivity triggers, including cold, hot, sugar, acid, and other energies or chemicals proximal to the sensitive pulp underlying the dentin, where they can cause pain or discomfort. That is, by expectorating, addition oxidizing agent is preserved for stain remediation, and the metal salt remaining to interact with the oxidizing agent is localized where it is most likely to provide additional benefits in the way of recalcification or sensitivity reduction when precipitated.

The bleaching composition may comprise an oxidizing or bleaching agent. Bleaching agents include peroxides, perborates, percarbonates, peroxyacids, persulfates, and combinations thereof. Suitable peroxide compounds include hydrogen peroxide, urea peroxide, calcium peroxide, sodium peroxide, zinc peroxide, or combinations thereof. One example of a percarbonate is sodium percarbonate. Exemplary persulfates include oxones. Some bleaching agents provide a burn sensation within an oral care composition, for example peroxides and percarbonates.

The compositions of the present invention may contain bleaching agents in an amount of from about 0.01% to about 30%, from about 0.1% to about 10%, or from about 0.5% to about 5%, by total weight of the oral care composition. To avoid the burning sensation that may occur with some bleaching agents, the amount of the bleaching agent used, if used, may be relatively low. One of ordinary skill will appreciate that a relatively low amount will vary with the delivery form of the bleaching composition. However, in some embodiments, it is desirable to have a bleaching agent, such as a peroxide, available to react with residual metal salts from the fluoride composition, even on or near the gums or soft tissues. This may help promote a pleasant taste and mouthfeel after using the bleaching composition, which, in turn, promotes compliance with the regimen.

It may be desirable to provide relatively lower doses of bleaching agent than are typically used. For example, if the bleaching composition is intended to be applied using a toothbrush, the amount of the bleaching agent may be limited to less than or equal to 5% of the bleaching composition, by weight of the bleaching composition. As another example, if the bleaching composition is delivered on a strip, the strip may be sized to cover at least a portion of the gums, and may comprise less than 16% of the bleaching agent, or less than 10% of the bleaching agent, or less than 6% of the bleaching agent, by weight of the composition adjacent to the gums. In some embodiments, the bleaching composition may be provided in a staged delivery form that provides a higher concentration of the bleaching agent to the teeth than to the gums.

The oxidizing or bleaching agent may provide whitening or stain reduction on the teeth. During the course of its usage, the oxidizing or bleaching agent may also precipitate trace amounts of residual metal salts or other ingredients from the fluoride composition. By precipitating those residual compounds, the oxidizing or bleaching agent may help to reduce any residual metallic, astringent, dry, or otherwise unpleasant organoleptic effects from the fluoride composition. This may be more noticeable to the user where the fluoride composition is not formulated with high levels of flavorants, rheology modifiers, sensates, etc. to overcome any unpleasant organoleptic effects.

The bleaching composition may further comprise flavorants, sensates, and the like to provide a pleasant taste and mouthfeel. In particular, the bleaching composition may provide a significantly more pleasant sensation after use than the fluoride composition. For example, if users are provided a scale of 1-5 for taste and mouth feel of the composition, with 5 being ideal, the bleaching composition may score, on average, at least 0.5 points higher than the fluoride composition. In some embodiments, the bleaching composition may score, on average, at least 3.5, or at least 4 on the 5-point scale. As discussed above, the second composition may diminish an unpleasant aftertaste or mouth feel from the first composition. For example, if users are provided a scale of 1-5 for taste and mouth feel of the composition, with 5 being ideal, a completed two-step regimen including sequential use of the first and second compositions, may score, on average, at least 0.5 points higher than the first composition alone. In some embodiments, a completed two-step regimen including sequential use of the first and second compositions may score, on average, at least 3.5, or at least 4 on the 5-point scale. Additives to improve taste and/or mouth feel, if used, may be selected and dosed to provide a pleasant taste and/or mouthfeel for some time after use. For example, if nothing else is put in the mouth after using the bleaching composition (excepting possibly a water rinse), the pleasant taste and/or mouth feel may persist for at least about 30 minutes, about 60 minutes, about 90 minutes, about 120 minutes, about 240 minutes, or even about 480 minutes. The smooth mouth feel may include a sensation described by a user as clean or slick or smooth, and may be facilitated both by cleaning benefits in the first and/or bleaching compositions (e.g., mechanical cleaning by abrasives, or chemical cleaning by the use of surfactants) and by components of the bleaching composition which may have the effect of increasing the feeling of moistness or smoothness in the mouth. For example, some rheology modifiers and/or polymeric additives may cling to the surface of the mouth and provide a moist, lubricious sensation, including the feeling on the tongue as the tongue is moved over the teeth and other surfaces in the mouth. An exemplary polymeric rheology modifier which may contribute to a smooth, lubricious mouth feel is a class of high molecular weight homo- and copolymers of acrylic acid crosslinked with a polyalkenyl polyether, commercially available under the name CARBOPOL (Lubrizol Corp., Wickliffe, Ohio, USA).

The bleaching composition may be used during the third stage. The third stage may have sub-stages. The third stage may comprise applying the bleaching composition to a dental hygiene device. The third stage may comprise using the dental hygiene device to apply the bleaching composition to the teeth and/or gums. The third stage may comprise expectorating. The third stage may comprise rinsing with water, a treatment rinse, or a mouthwash. If a rinsing step is used, it may be the final step.

A process for using a dentifrice with incremental chemistry is also provided. The process may comprise applying a fluoride composition to the teeth and/or gums. The process may comprise expectorating. The process may exclude rinsing after the use of the fluoride composition and before the use of the bleaching composition. The process may comprise applying a bleaching composition to the teeth and/or gums. The process may comprise expectorating. The process may comprise rinsing after expectorating the bleaching composition. The first and/or bleaching composition can be applied to the teeth and/or gums in any suitable manner. In some embodiments, a user dispenses the first and/or bleaching composition onto a toothbrush and proceeds with applying the composition to the oral cavity as part of a brushing regimen. In some embodiments, the composition, or each composition, is used for about one minute. In some embodiments, the bleaching composition is applied to a toothbrush or the oral cavity within about 5, 10, 15, 30, 45, 60, 120, 180, 240, 300, 360, or 420 seconds of the first component being applied to a toothbrush or the oral cavity. The toothbrush may be a manual toothbrush or a power toothbrush, having bristles which are very soft, soft, medium, firm, or very firm. In some embodiments, the fluoride chemistry can be applied using a power brush and the bleaching chemistry can be applied using a manual brush, or vice versa. In some embodiments, the first and/or bleaching composition is applied using an applicator strip or tray. The user may load the composition onto the strip or tray before applying the strip or tray to the mouth, or the strip or tray may come pre-loaded with the composition. Other possible dental hygiene devices include syringes, tubes, swabs, puffs, cups, and the like, which may be used to introduce an oral care composition into the oral cavity.

The first or bleaching composition may comprise a variety of oral care ingredients, for oral health, cosmetic, or sensory benefits, or to provide a stable, homogenous composition. Exemplary ingredients include, without limitation, sweeteners, carrier materials, antimicrobial agents, surfactants, flavors, anti-tartar agents, colorants, sensates, abrasives, thickening material or binders, humectants, and combinations thereof.

Sweeteners include saccharin, chloro-sucrose (sucralose), steviolglycosides, rebaudioside A, rebaudioside B, rebaudioside C, rebaudioside D, rebaudioside E, rebaudioside F, dulcoside A, dulcoside B, rubusoside, stevia, stevioside, acesulfame K, xylitol, neohesperidine DC, alitame, aspartame, neotame, alitame, thaumatin, cyclamate, glycyrrhizin, mogroside IV, mogroside V,

Luo Han Guo sweetener, siamenoside, monatin and its salts (monatin SS, RR, RS, SR), curculin, monellin, mabinlin, brazzein, hemandulcin, phyllodulcin, glycyphyllin, phloridzin, trilobatin, baiyanoside, osladin, polypodoside A, pterocaryoside A, pterocaryoside B, mukurozioside, phlomisoside I, periandrin I, abrusoside A, cyclocarioside I,N-[N-[3-(3-hydroxy-4-methoxyphenyl)propyl]-L-α-aspartyl]-L-phenylalanine 1-methyl ester, N-[N-[3-(3-hydroxy-4-methoxyphenyl)-3-methylbutyl]-L-α-aspartyl]-L-phenylalanine 1-methyl ester, N-[N-[3-(3-methoxy-4-hydroxyphenyl)propyl]-L-α-aspartyl]-L-phenylalanine 1-methyl ester, salts thereof, and combinations thereof.

Rebiana can be a steviolglycoside from Cargill Corp., Minneapolis, MN, which is an extract from the leaves of the Stevia rebaudiana plant (hereinafter referred to as “Rebiana”). This is a crystalline diterpene glycoside, about 300x sweeter than sucrose. Examples of suitable stevioglycosides which may be combined include rebaudioside A, rebaudioside B, rebaudioside C, rebaudioside D, rebaudioside E, rebaudioside F, dulcoside A, dulcoside B, rubusoside, stevioside, or steviolbioside. According to particularly desirable examples of the present invention, the combination of high-potency sweeteners comprises rebaudioside A in combination with rebaudioside B, rebaudioside C, rebaudioside F, rebaudioside F, stevioside, steviolbioside, dulcoside A.

Carrier materials include water, glycerin, sorbitol, polyethylene glycols having a molecular weight of less than about 50,000, propylene glycol and other edible polyhydric alcohols, ethanol, or combinations thereof. The oral care compositions of the present invention include from about 5% to about 80%, by weight of the composition, of a carrier material. In certain examples, the compositions contain carrier materials in an amount of from about 10% to about 40%, by total weight of the oral care composition.

Antimicrobial agents include quaternary ammonium compounds. Those useful in the present invention include, for example, those in which one or two of the substitutes on the quaternary nitrogen has a carbon chain length (typically alkyl group) from about 8 to about 20, typically from about 10 to about 18 carbon atoms while the remaining substitutes (typically alkyl or benzyl group) have a lower number of carbon atoms, such as from about 1 to about 7 carbon atoms, typically methyl or ethyl groups. Dodecyl trimethyl ammonium bromide, tetradecylpyridinium chloride, domiphen bromide, N-tetradecyl-4-ethyl pyridinium chloride, dodecyl dimethyl (2-phenoxyethyl) ammonium bromide, benzyl dimethoylstearyl ammonium chloride, quaternized 5-amino-1,3-bis(2-ethyl-hexyl)-5-methyl hexahydropyrimidine, benzalkonium chloride, benzethonium chloride and methyl benzethonium chloride are exemplary of typical quaternary ammonium antibacterial agents.

Other quaternary ammonium compounds include the pyridinium compounds. Examples of pyridinium quaternary ammonium compounds include bis[4-(R-amino)-1-pyridinium] alkanes as disclosed in U.S. Pat. No. 4,206,215, Jun. 3, 1980, to Bailey and cetylpyridinium and tetradecylpyridinium halide salts (i.e., chloride, bromide, fluoride and iodide).

The oral care compositions may also include other antimicrobial agents including non-cationic antimicrobial agents such as halogenated diphenyl ethers, phenolic compounds including phenol and its homologs, mono and poly-alkyl and aromatic halophenols, resorcinol and its derivatives, xylitol, bisphenolic compounds and halogenated salicylanilides, benzoic esters, and halogenated carbanilides. Also useful antimicrobials are enzymes, including endoglycosidase, papain, dextranase, mutanase, and combinations thereof. Such agents are disclosed in U.S. Pat. No. 2,946,725, Jul. 26, 1960, to Norris et al. and in U.S. Pat. No. 4,051,234 to Gieske et al. Examples of other antimicrobial agents include chlorhexidine, and flavor oils such as thymol. In another example, the antimicrobial agent can include triclosan.

The compositions of the present invention may contain antimicrobial agents in an amount of from about 0.035% or more, from about 0.1% to about 1.5%, from about 0.045% to about 1.0%, or from about 0.05% to about 0.10%, by total weight of the oral care composition.

Surfactants may include anionic surfactants such as organophosphate, which include alkyl phosphates. These surface active organophosphate agents have a strong affinity for enamel surface and have sufficient surface binding propensity to desorb pellicle proteins and remain affixed to enamel surfaces. Suitable examples of organophosphate compounds include mono-, di- or triesters represented by the general structure below wherein Z1, Z2, or Z3 may be identical or different, at least one being an organic moiety, in one example selected from linear or branched, alkyl or alkenyl group of from 1 to 22 carbon atoms, optionally substituted by one or more phosphate groups; alkoxylated alkyl or alkenyl, (poly)saccharide, polyol or polyether group.

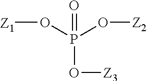

Some other organophosphate agents include alkyl or alkenyl phosphate esters represented by the following structure:

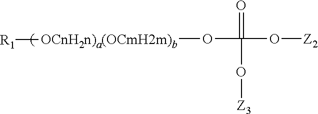

wherein R1 represents a linear or branched, alkyl or alkenyl group of from 6 to 22 carbon atoms, optionally substituted by one or more phosphate groups; n and m, are individually and separately, 2 to 4, and a and b, individually and separately, are 0 to 20; Z2 and Z3 may be identical or different, each represents hydrogen, alkali metal, ammonium, protonated alkyl amine or protonated functional alkyl amine such as an alkanolamine, or a R1—(OCnH2n)a(OCmH2m)b—group. Examples of suitable agents include alkyl and alkyl (poly)alkoxy phosphates such as lauryl phosphate; PPGS ceteareth-10 phosphate; Laureth-1 phosphate; Laureth-3 phosphate; Laureth-9 phosphate; Trilaureth-4 phosphate; C12-18 PEG 9 phosphate; Sodium dilaureth-10 phosphate. In one example, the alkyl phosphate is polymeric. Examples of polymeric alkyl phosphates include those containing repeating alkoxy groups as the polymeric portion, in particular 3 or more ethoxy, propoxy isopropoxy or butoxy groups.

Zwitterionic or amphoteric surfactants useful in the present invention can include derivatives of aliphatic quaternary ammonium, phosphonium, and sulfonium compounds, in which the aliphatic radicals can be straight chain or branched, and wherein one of the aliphatic substituents contains from about 8 to 18 carbon atoms and one contains an anionic water-solubilizing group, such as carboxy, sulfonate, sulfate, phosphate or phosphonate. Suitable amphoteric surfactants include betaine surfactants such as disclosed in U.S. Pat. No. 5,180,577 to Polefka et al. Typical alkyl dimethyl betaines include decyl betaine or 2-(N-decyl-N,N-dimethylammonio)acetate, coco betaine or 2-(N-coco-N,N-dimethyl ammonio) acetate, myristyl betaine, palmityl betaine, lauryl betaine, cetyl betaine, stearyl betaine, etc. Amphoteric surfactants useful herein further include amine oxide surfactants. The amidobetaines are exemplified by cocoamidoethyl betaine, cocamidopropyl betaine (CAPB), and lauramidopropyl betaine. The unwanted tastes often associated with these surfactants are soapy, bitter, chemical, or artificial.

Additional suitable polymeric organophosphate agents can include dextran phosphate, polyglucoside phosphate, alkyl polyglucoside phosphate, polyglyceryl phosphate, alkyl polyglyceryl phosphate, polyether phosphates and alkoxylated polyol phosphates. Some specific examples are PEG phosphate, PPG phosphate, alkyl PPG phosphate, PEG/PPG phosphate, alkyl PEG/PPG phosphate, PEG/PPG/PEG phosphate, dipropylene glycol phosphate, PEG glyceryl phosphate, PBG (polybutylene glycol) phosphate, PEG cyclodextrin phosphate, PEG sorbitan phosphate, PEG alkyl sorbitan phosphate, and PEG methyl glucoside phosphate. Suitable non-polymeric phosphates include alkyl mono glyceride phosphate, alkyl sorbitan phosphate, alkyl methyl glucoside phosphate, alkyl sucrose phosphates. The impurities in these phosphates may induce a burning sensation Impurities may include dodecanol, dodecanal, benzaldehyde, and other TRPA1 or TRPV1 agonists.

Cationic surfactants useful in the present invention can include derivatives of quaternary ammonium compounds having one long alkyl chain containing from about 8 to 18 carbon atoms such as lauryl trimethylammonium chloride, cetyl trimethylammonium bromide, coconut alkyltrimethylammonium nitrite, cetyl pyridinium fluoride, etc. Quaternary ammonium halides having detergent properties can be used, such as those described in U.S. Pat. No. 3,535,421 to Briner et al. Certain cationic surfactants can also act as germicides in the oral care compositions disclosed herein.

Examples of some flavors and flavor components that may be used in oral care compositions are mint oils, wintergreen, clove bud oil, cassia, sage, parsley oil, marjoram, lemon, orange, propenyl guaethol, heliotropine, 4-cis-heptenal, diacetyl, methyl-p-tert-butyl phenyl acetate, methyl salicylate, ethyl salicylate, 1-menthyl acetate, oxanone, a-irisone, methyl cinnamate, ethyl cinnamate, butyl cinnamate, ethyl butyrate, ethyl acetate, methyl anthranilate, iso-amyl acetate, iso-amyl butyrate, allyl caproate, eugenol, eucalyptol, thymol, cinnamic alcohol, octanol, octanal, decanol, decanal, phenylethyl alcohol, benzyl alcohol, a-terpineol, linalool, limonene, citral, neral, geranial, geraniol nerol, maltol, ethyl maltol, anethole, dihydroanethole, carvone, menthone, β-damascenone, ionone, γ-decalactone, γ-nonalactone, γ-undecalactone, or combinations thereof. Generally suitable flavoring ingredients are chemicals with structural features and functional groups that are less prone to redox reactions. These include derivatives of flavor chemicals that are saturated or contain stable aromatic rings or ester groups.

Anti-tartar agents include pyrophosphate salts as a source of pyrophosphate ion. The pyrophosphate salts useful in the present compositions include, for example, the mono-, di- and tetraalkali metal pyrophosphate salts and combinations thereof. Disodium dihydrogen pyrophosphate (Na2H2P2O7), sodium acid pyrophosphate, tetrasodium pyrophosphate (Na4P2O7), and tetrapotassium pyrophosphate (K4P2O7) in their unhydrated as well as hydrated forms are further species. In compositions of the present invention, the pyrophosphate salt may be present in one of three ways: predominately dissolved, predominately undissolved, or a combination of dissolved and undissolved pyrophosphate. The amount of pyrophosphate salt useful in making these compositions is any tartar control effective amount. In varying examples, the amount of pyrophosphate salt may be from about 1.5% to about 15%, from about 2% to about 10%, or about 3% to about 8%, by total weight of the oral care composition. Examples of some colorants that may be used in oral care compositions include D&C Yellow No. 10, FD&C Blue No. 1, FD&C Red No. 40, D&C Red No. 33 and combinations thereof. In certain examples, the composition comprises colorant in an amount of from about 0.0001% to about 0.1% or from about 0.001% to about 0.01%, by weight of the oral care composition. Some colorants provide an unwanted taste, for example, D&C Red No. 33. The unwanted tastes often associated with this colorant are metallic, sharp, or chemical. Colorants are generally present in an amount of from about 0.001% to about 0.5%, by weight of the oral care composition.

Sensates may also be part of an oral care composition. Sensate molecules such as cooling, warming, and tingling agents are useful to deliver signals to the user. Sensates are generally present in an amount of from about 0.001% to about 0.8%, by weight of the oral care composition. The most well-known cooling sensate compound can be menthol, particularly L-menthol, which is found naturally in peppermint oil notably of Mentha arvensis L and Mentha viridis L. Other isomers of menthol (neomenthol, isomenthol and neoisomenthol) have somewhat similar, but not identical odor and taste, for instance having disagreeable odor and taste described as earthy, camphor, musty, etc. The biggest difference among the isomers is in their cooling potency. L-menthol provides the most potent cooling, by having the lowest cooling threshold of about 800 ppb, which is the concentration level where the cooling effect can be clearly recognized. At this level, there can be no cooling effect for the other isomers. For example, d-neomenthol is reported to have a cooling threshold of about 25,000 ppb and 1-neomenthol about 3,000 ppb.

Of the menthol isomers the 1-isomer occurs most widely in nature and is typically what is referred by the name menthol having coolant properties. L-menthol has the characteristic peppermint odor, has a clean fresh taste and exerts a cooling sensation when applied to the skin and mucosal surfaces.

Among synthetic coolants, many are derivatives of or are structurally related to menthol, for example containing the cyclohexane moiety, and derivatized with functional groups including carboxamide, ketal, ester, ether and alcohol. Examples include the p-menthanecarboxamide compounds such as N-ethyl-p-menthan-3-carboxamide, known commercially as “WS-3”, and others in the series such as WS-5 (N-ethoxycarbonylmethyl-p-menthan-3-carboxamide), WS-12 (1R* ,2S *)-N-(4-Methoxyphenyl)-5-methyl-2-(1-methylethyl)cyclohexanecarboxamidel and WS-14 (N-tert-butyl-p-menthan-3-carboxamide). Examples of menthane carboxy esters include WS-4 and WS-30. An example of a synthetic carboxamide coolant that is structurally unrelated to menthol is N,2,3-trimethyl-2-isopropylbutanamide, known as “WS-23”. Additional examples of synthetic coolants include alcohol derivatives such as 3-(1-menthoxy)-propane-1,2-diol known as TK-10, isopulegol (under the tradename Coolact P) and p-menthane-3,8-diol (under the tradename Coolact 38D) all available from Takasago Corp., Tokyo, Japan; menthone glycerol acetal known as MGA; menthyl esters such as menthyl acetate, menthyl acetoacetate, menthyl lactate known as Frescolat® supplied by Symrise AG, Holzminden, Germany, and monomenthyl succinate under the tradename Physcool from V. Mane FILS, Notre Dame, France. TK-10 is described in U.S. Pat. No. 4,459,425 to Amano et al. Other alcohol and ether derivatives of menthol are described in GB 1,315,626 and in U.S. Pat. Nos. 4,029,759; 5,608,119; and 6,956,139. WS-3 and other carboxamide cooling agents are described in U.S. Pat. Nos. 4,136,163; 4,150,052; 4,153,679; 4,157,384; 4,178,459 and 4,230,688.

Additional N-substituted p-menthane carboxamides are described in WO 2005/049553A1 including N-(4-cyanomethylphenyl)-p-menthanecarboxamide, N-(4-sulfamoylphenyl)-p-menthanecarboxamide, N-(4-cyanophenyl)p-menthanecarboxamide, N-(4-acetylphenyl)-p-menthanecarboxamide, N-(4-hydroxymethylphenyl)-p-menthanecarboxamide and N-(3-hydroxy-4-methoxyphenyl)-p-menthanecarboxamide. Other N-substituted p-menthane carboxamides include amino acid derivatives such as those disclosed in WO 2006/103401 and in U.S. Pat. Nos. 4,136,163; 4,178,459 and 7,189,760 such as N-((5-methyl-2-(1-methylethyl)cyclohexyl)carbonyl)glycine ethyl ester and N4(5-methyl-2-(1-methylethyl)cyclohexyl)carbonyl) alanine ethyl ester. Menthyl esters including those of amino acids such as glycine and alanine are disclosed e.g., in EP 310,299 and in U.S. Pat. Nos. 3,917,613; 3,991,178; 5,703,123; 5,725,865; 5,843,466; 6,365,215; and 6,884,903. Ketal derivatives are described, e.g., in U.S. Pat. Nos. 5,266,592; 5,977,166; and 5,451,404. Additional agents that are structurally unrelated to menthol but have been reported to have a similar physiological cooling effect include alpha-keto enamine derivatives described in U.S. Pat. No. 6,592,884 including 3-methyl-2-(1-pyrrolidinyl)-2-cyclopenten-1-one (3-MPC), 5-methyl-2-(1-pyrrolidinyl)-2-cyclopenten-1-one (5-MPC), and 2,5-dimethyl-4-(1-pyrrolidinyl)-3(2H)-furanone (DMPF); icilin (also known as AG-3-5, chemical name 1-[2-hydroxyphenyl]-4-[2-nitrophenyl]-1,2,3,6-tetrahydropyrimidine-2-one) described in Wei et al., J. Pharm. Pharmacol. (1983), 35:110-112. Reviews on the coolant activity of menthol and synthetic coolants include H. R. Watson, et al. J. Soc. Cosmet. Chem. (1978), 29, 185-200 and R. Eccles, J. Pharm. Pharmacol., (1994), 46, 618-630.

Additional agents that are structurally unrelated to menthol but have been reported to have a similar physiological cooling effect include alpha-keto enamine derivatives described in U.S. Pat. No. 6,592,884 including 3-methyl-2-(1-pyrrolidinyl)-2-cyclopenten-1-one (3-MPC), 5-methyl-2-(1-pyrrolidinyl)-2-cyclopenten-1-one (5-MPC), and 2,5-dimethyl-4-(1-pyrrolidinyl)-3(2H)-furanone (DMPF); icilin (also known as AG-3-5, chemical name 1-[2-hydroxyphenyl]-4-[2-nitrophenyl]-1,2,3,6-tetrahydropyrimidine-2-one) described in Wei et al., J. Pharm. Pharmacol. (1983), 35:110-112 and phosphine oxides as reported in U.S. Pat. No. 4,070,496.

Some examples of warming sensates include ethanol; capsicum; nicotinate esters, such as benzyl nicotinate; polyhydric alcohols; capsicum powder; a capsicum tincture; capsicum extract; capsaicin; homocapsaicin; homodihydrocapsaicin; nonanoyl vanillyl amide; nonanoic acid vanillyl ether; vanillyl alcohol alkyl ether derivatives such as vanillyl ethyl ether, vanillyl butyl ether, vanillyl pentyl ether, and vanillyl hexyl ether; isovanillyl alcohol alkyl ethers; ethylvanillyl alcohol alkyl ethers; veratryl alcohol derivatives; substituted benzyl alcohol derivatives; substituted benzyl alcohol alkyl ethers; vanillin propylene glycol acetal; ethylvanillin propylene glycol acetal; ginger extract; ginger oil; gingerol; zingerone; or combinations thereof. Warming sensates are generally included in an oral care composition at a level of about 0.05% to about 2%, by weight of the oral care composition.

Abrasive polishing material can be any material that does not excessively abrade dentin. The oral care compositions of the present invention may comprise abrasive polishing material in an amount of from about 6% to about 70% or from about 10% to about 50%, by weight of the oral care composition. Typical abrasive polishing materials include silicas including gels and precipitates; aluminas; phosphates including orthophosphates, polymetaphosphates, and pyrophosphates; and mixtures thereof. Specific examples include dicalcium orthophosphate dihydrate, calcium pyrophosphate, tricalcium phosphate, calcium polymetaphosphate, insoluble sodium polymetaphosphate, rice hull silica, hydrated alumina, beta calcium pyrophosphate, calcium carbonate, and resinous abrasive materials such as particulate condensation products of urea and formaldehyde, and others such as disclosed by Cooley et al in U.S. Pat. No. 3,070,510. In certain examples, if the oral composition or particular phase comprises a polyphosphate having an average chain length of about 4 or more, calcium containing abrasives and alumina are not preferred abrasives.

Silica dental abrasives of various types are often used in oral care compositions due to their exceptional dental cleaning and polishing performance without unduly abrading tooth enamel or dentine. Silica abrasive polishing materials that may be used in the present invention, as well as other abrasives, generally have an average particle size ranging between about 0.1 to about 30 um or from about 5 to about 15 um. The abrasive can be precipitated silica or silica gels such as the silica xerogels described in Pader et al., U.S. Pat. No. 3,538,230 and DiGiulio, U.S. Pat. No. 3,862,307. Silica xerogels marketed under the trade name “Syloid” by the W.R. Grace & Company, Davison Chemical Division, Augusta, GA may be used. Also precipitated silica materials such as those marketed by the J. M. Huber Corporation, Edison, NJ under the trade name, “Zeodent”, particularly the silica carrying the designation “Zeodent 119”, may be used. The types of silica dental abrasives useful in the oral care compositions of the present invention are described in more detail in Wason, U.S. Pat. No. 4,340,583; and Rice U.S. Pat. Nos/ 5,589,160; 5,603,920; 5,651,958; 5,658,553; and 5,716,601.

Thickening material or binders may be used to provide a desirable consistency to the oral care compositions of the present invention. For example when the oral care compositions are in the form of dentifrices, topical oral gels, mouthrinse, denture product, mouthsprays, lozenges, oral tablets or chewing gums, the amount and type of the thickening material will depend upon the form of the product. Thickening materials include carboxyvinyl polymers, carrageenan, hydroxyethyl cellulose, and water soluble salts of cellulose ethers such as sodium carboxymethylcellulose and sodium hydroxyethyl cellulose. Natural gums such as gum karaya, xanthan gum, gum arabic, and gum tragacanth can also be used. Colloidal magnesium aluminum silicate or finely divided silica can be used as part of the thickening material to further improve texture. Thickening materials can be used in an amount from about 0.1% to about 15%, by weight of the oral care composition.

Humectants keep oral care compositions from hardening upon exposure to air and certain humectants can also impart desirable sweetness of flavor to dentifrice compositions. Suitable humectants for use in the present invention include glycerin, sorbitol, polyethylene glycol, propylene glycol, xylitol, and other edible polyhydric alcohols. The oral care compositions of the present invention may comprise humectants in an amount of from about 0% to about 70% or from about 15% to about 55%, by weight of the oral care composition.

### EXAMPLES

### Example 1

### Reduction of Oral Malador using a Dentifrice with Incremental Chemistries

This example demonstrates the reduction of malador using a dentifrice with incremental chemistries compared to a control paste. The test dentifrice included a first container with a 0.45% stannous fluoride paste and a second container with a 3% hydrogen peroxide paste, along with a manual, soft toothbrush. The control dentifrice was a commercially available 0.243% sodium fluoride paste, along with a manual, soft toothbrush.

A randomized, single-blind, 2 treatment cross-over study with 4 periods was performed. Twenty-four healthy adult volunteers with evidence of reproducible malodor completed the study. Subjects completed a one week acclimation period, followed by a 24 hour treatment period and then an at least 1 week wash-out period. Halimeter breath measurements were taken at Baseline, 3 and 24 hours after morning brushing. The treatment and wash-out periods were repeated until 4 treatment periods were complete. Halimeter Breath Measurement

Subjects were assessed for volatile sulfur compound emissions (VSC) utilizing a commercially available portable instrument called a Halimeter (Interscan Corporation, CA). This instrument was sensitive to hydrogen sulfide and methyl mercaptan, two of the primary components of foul breath odor. A trained technician performed all Halimeter measurements. Subjects were instructed to keep their mouth closed for 2 minutes. Subjects then placed a piece of barrier tape on the halimeter board above the hole and then placed one end of a clean paper cylinder through the hole in the halimeter board. Subjects were instructed to swallow, if they would like, 30-45 seconds prior to their halimeter measurement, being sure to keep their mouth closed. After 2 minutes had elapsed, subjects were instructed to inhale through their nose and hold their breath. The technician recorded a background halimeter value immediately before the subject approached the halimeter. The subject then approached the halimeter and, while holding their breath, placed their teeth and lips loosely around the tube; the subject's tongue was under the tube and their nose was touching the board. While the subject held their breath, the instrument drew air from the mouth (without touching the subject's mouth) and the technician recorded the measured value indicated on the instrument. The subject then removed the barrier tape and the paper cylinder they used and discarded them in the appropriate receptacle.

After the baseline halimeter measurement, subjects brushed with the products, and a halimeter measurement was taken 3 hours after the AM brushing. The night before the 24-h study visit, subjects were instructed to brush with the products prior to 11:00 PM; they were instructed to abstain from tongue brushing.

For the control group, subjects were instructed to brush thoroughly with the products provided. For the experimental group, subjects were instructed to brush the whole mouth with a full brush head of the experimental 0.45% stannous fluoride paste a for 1 minute and expectorate, but not to rinse with water; they were instructed to abstain from tongue brushing. Subjects were instructed to then brush the whole mouth with a full brush head of the experimental 3% hydrogen peroxide paste for 1 minute and expectorate; they were instructed to abstain from tongue brushing. Subjects were instructed to time brushing with the timer provided. Subjects were instructed to rinse with tap water following the use of the experimental 3% hydrogen peroxide paste.

### Results

The experimental dentifrice with incremental chemistries demonstrated significantly (p<0.0001) lower mean VSC by 34% relative to the control at each of the 3 hour and 24 hour visits.

### Example 2

### Plaque Removal Efficacy using a Dentifrice with Incremental Chemistries

This example demonstrates the plaque removal efficacy using a dentifrice with incremental chemistries compared to a paste containing sodium fluoride and triclosan. The experimental dentifrice included a first container with a 0.45% stannous fluoride paste and a second container with a 3% hydrogen peroxide paste, along with an Oral-B Sensitive Advantage extra soft, manual toothbrush. The control dentifrice was a commercially available paste containing 0.24% sodium fluoride and 0.30% triclosan, along with an Oral-B Sensitive Advantage extra soft, manual toothbrush. Subjects were acclimated prior to the test period using a commercially available 0.76% sodium monofluorophosphate paste and an Oral-B Sensitive Advantage extra soft, manual toothbrush.

A randomized, controlled, double-blind, 2-treatment parallel group study was performed. Forty-eight healthy adult volunteers with plaque completed the study. Overnight (pre-brush) plaque and post-brush plaque were measured by digital image analysis of fluorescein-disclosed plaque.

An acclimation visit preceded the baseline visit. During the acclimation visit, subjects were instructed to brush thoroughly, twice daily. Subjects were instructed to rinse with water after brushing. During the treatment period, the control group was instructed to brush thoroughly, twice daily. They were instructed to rinse with water after brushing. Subjects in the test group were instructed to apply enough of the experimental 0.45% stannous fluoride paste onto the toothbrush to cover the bristles and to brush thoroughly for 1 minute. After brushing with the experimental 0.45% stannous fluoride paste, subjects expectorated but were instructed not to rinse with water. Subjects were then instructed to apply enough of the 3% hydrogen peroxide paste onto the toothbrush to cover the bristles and brush thoroughly, for at least 1 minute. Subjects were instructed to rinse with tap water following the use of the experimental 3% hydrogen peroxide paste. Subjects in the test group were instructed to brush twice daily. For the duration of the study, all subjects were instructed not to use any non-study oral hygiene products. Fluorescein Plaque Disclosing Procedure

Subjects rinsed for 10 seconds with 25 ml of phosphate buffer; then rinsed for 1 minute with 5.0 ml of 1240 ppm fluorescein in phosphate buffer. Subjects then rinsed 3 times for 10 seconds with 25 ml of phosphate buffer.

### Digital Plaque Imaging Camera

Digital Plaque Imaging was conducted using ASTM E2670-09. The photographic system consisted of a high resolution digital camera equipped with a 25 mm lens and a linear polarizer to permit cross-polarized light. A UV flash provided the lighting. The unit was connected to a personal computer, which recorded and analyzed the images. Prior to daily use, the system was standardized to assure proper operation. Additionally, a color standard was centered and imaged every half hour, then removed prior to imaging subjects. A digital image of the maxillary and mandibular anterior facial surfaces was captured. Tooth and plaque pixels were classified in the digital image and the percent plaque coverage on the teeth was calculated. For each examination period, lighting in the exam room was background or ambient. The subject sat on a stool in front of a chin rest used to hold the head still. The subject placed his/her chin on the chin rest, then positioned two plastic retractors into the mouth to retract his/her lips and cheeks. It was also acceptable for the subject to position the retractors, then place his/her chin on the chin rest. The subject was instructed to use the retractors to retract his/her lips and cheek (toward the ears) as far as possible. The incisal edges of the front teeth were placed together and centered in the camera. The chin rest could be adjusted to bring the teeth into the plane of focus and ensure the image was centered. Prior to exposure, the subject was instructed to draw air through their teeth and to position their tongue away from the teeth so that the tongue was not visible. By proper positioning of the camera, frontal images of each subject were taken at each visit.

### Results

The experimental dentifrice with incremental chemistries demonstrated significantly (p<0.02) less mean plaque area coverage relative to the control for both visits (Week 1 and Week 3) as well as each time point (pre-brush and post-brush). Averaging Weeks 1 and 3 visits, percent reductions in mean plaque area coverage for the experimental group relative to control were 56% for pre-brush and 35% for postbrush.

### Example 3

### Plaque Removal Efficacy using a Dentifrice with Incremental Chemistries

This example demonstrates the plaque removal efficacy using a dentifrice with incremental chemistries compared to a control paste. The test dentifrice included a first container with a 0.45% stannous fluoride paste and a second container with a 3% hydrogen peroxide paste, along with an Oral-B Sensitive Advantage extra soft, manual toothbrush. The control dentifrice was a commercially available paste containing 0.76% sodium monolluorophospate, along with an Oral-B Sensitive Advantage extra soft, manual toothbrush. Subjects were acclimated prior to the test period using a commercially available 0.76% sodium monofluorophosphate paste and an Oral-B Sensitive Advantage extra soft, manual toothbrush.

A randomized, controlled, double-blind, 2-treatment parallel group study was performed as described in Example 2. Forty-five healthy adult volunteers with plaque completed the study. Overnight (pre-brush) plaque and post-brush plaque were measured by digital image analysis of fluorescein-disclosed plaque as described in Example 2.

### Results

Averaging the Weeks 1 and 3 visits, the experimental group exhibited a significantly (p<0.03) less mean plaque area relative to the control at both the pre-brush and post-brush time points. The percent reduction in mean plaque area for the experimental group relative to the control group was 36% for the average of the Weeks 1 and 3 pre-brush visits. The percent reduction in mean plaque area for the experimental group relative to the control group was 30% for the average of the Weeks 1 and 3 post-brush visits.

### Example 4

### Gingivitis and Plaque Efficacy using a Dentifrice with Incremental Chemistries

This example demonstrates the gingivitis and plaque efficacy using a dentifrice with incremental chemistries compared to a control paste. The experimental dentifrice included a first container with a 0.45% stannous fluoride paste and a second container with a 3% hydrogen peroxide paste, along with an Oral-B Sensitive Advantage extra soft, manual toothbrush. The control dentifrice was a commercially available paste containing 0.76% sodium monolluorophospate, along with an Oral-B Sensitive Advantage extra soft, manual toothbrush.

A randomized, controlled, double-blind, 2-treatment parallel group study with 5 visits was performed. Eighty-four healthy adult volunteers with plaque and gingivitis completed the study.

Subjects brushed their teeth the night before each study visit. At baseline, week 5, and week 11 visits, a comprehensive oral examination was conducted to evaluate the oral and perioral region, including hard and soft tissues. A trained evaluator measured gingival bleeding. Next, subjects had plaque disclosed with a red disclosing solution, and a trained evaluator examined the subjects for plaque.

### Results

At week 5 and week 11, the experimental group demonstrated significant (p<0.001) reductions in gingivitis, number of bleeding sites, and plaque relative to baseline, as well as significantly less mean gingivitis (p<0.02), mean number of bleeding sites (p<0.02), and mean plaque (p<0.05) relative to the control group.

### Example 5

### Example Dentrifrice with Incremental Chemistries

### Fluoride Chemistry—First Stage

| | |
|---|---|
| Stannous Fluoride, USP | 0.454 |
| Water | 2.600 |
| Glycerin, USP (99.7%) | 58.977 |
| Zinc Lactate Dihydrate (100%) | 2.500 |
| Sodium Phosphate Tribasic | 1.100 |
| Dodecahydrate | |
| Sodium Gluconate, USP | 0.652 |
| Sodium Hydroxide (50% solution) | 0.087 |
| Xanthan Gum, NF | 0.400 |
| Sodium Carboxymethylcellulose | 0.200 |
| (7M8SF)¹ | |
| Thickening Silica (Zeodent ® 165) ² | 1.500 |
| Silica (Zeodent ® 109) ² | 12.500 |
| Silica (Zeodent ® 119) ² | 12.500 |
| Sodium Lauryl Sulfate (28% solution), | 4.000 |
| Saccharin Sodium, USP (Granular), | 0.500 |
| Flavor | 1.030 |
| Colorants | 1.000 |

¹Available from Aqualon ® (Wilmington, Delaware, USA)
² Available from the J. M. Huber Corporation (Edison, New Jersey, USA)

### Bleaching Composition

| | |
|---|---|
| Hydrogen Peroxide (35%) | 8.700 |
| Glycerin, USP | 20.000 |
| Water | 65.400 |
| Sodium Acid Pyrophosphate | 1.000 |
| Carbopol ® 956 Polymer³ (CAS# is | 2.000 |
| 134499-38-0) | |
| Sodium Hydroxide (50% solution) | 0.900 |
| Saccharin Sodium, USP (Granular) | 0.500 |
| Flavor | 1.000 |
| Sucralose, USP | 0.500 |

³Available from the Goodrich Corporation (Akron, Ohio, USA)

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm ”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular examples of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A dentifrice composition comprising a multi-stage composition adapted to provide incremental chemistries during use, the multi-stage composition having a first stage comprising a fluoride agent, a second stage comprising a residue of the fluoride agent obtained after application and expectoration of the first stage, and a third stage comprising a mixture of the residue of the fluoride agent and a bleaching agent, wherein the bleaching agent is added to the fluoride agent after the expectoration of the first stage.

2. The dentifrice composition of claim 1, wherein the fluoride agent is stannous fluoride.

3. The dentifrice composition of claim 1, wherein the composition provides anti-plaque benefits overnight.

4. The dentifrice composition of claim 1, wherein the first stage is contained in a first container with a first shape and the third stage is contained in a second container with a second shape that is different from the first shape.

5. The dentifrice composition of claim 1, wherein the fluoride agent is applied for a duration from about 30 seconds to about 2 minutes.

6. The dentifrice composition of claim 1, wherein the bleaching agent is peroxide.

7. The dentifrice composition of claim 1, wherein the bleaching agent is applied less than one minute after expectoration of the fluoride agent.

8. The dentifrice composition of claim 3, wherein the composition reduces plaque formation for twenty-four hours after application.

9. The dentifrice composition of claim 1, wherein the first stage has staining activity.

10. A dentifrice composition comprising a multi-stage composition having incompatible first and second effective agents;
the first effective agent comprising a fluoride agent, the first effective agent being contained in a first container and being applied to a user's mouth and expectorated to leave a fluoride residue; and
the second effective agent comprising a bleaching agent, the second effective agent being contained in a second, separate container, the second effective agent applied to a user's mouth to mix with the fluoride residue; wherein the fluoride reside is detectable during application of the second effective agent.

11. The dentifrice composition of claim 10, wherein the fluoride agent is stannous fluoride.

12. The dentifrice composition of claim 10, wherein the composition provides anti-plaque benefits overnight.

13. The dentifrice composition of claim 10, wherein the fluoride agent is applied for a duration from about 30 seconds to about 2 minutes.

14. The dentifrice composition of claim 10, wherein the bleaching agent is peroxide.

15. A dentifrice composition comprising a mixture of a residue of an unmitigated fluoride agent and a dose of a bleaching agent, wherein the residue is obtained after a dose of the fluoride agent is applied to the teeth, brushed with a toothbrush, and expectorated without rinsing.

16. The dentifrice composition of claim 15, wherein the fluoride agent is stannous fluoride.

17. The dentifrice composition of claim 15, wherein the composition provides anti-plaque benefits overnight.

18. The dentifrice composition of claim 15, wherein the fluoride agent is applied for a duration from about 30 seconds to about 2 minutes.

19. The dentifrice composition of claim 15, wherein the bleaching agent is peroxide.

20. The dentifrice composition of claim 15, wherein the fluoride agent is contained in a first container with a first shape and the bleaching agent is contained in a second container with a second shape that is different from the first shape.
